(19) United States
(12) Patent Application Publication
(10) Pub. No.: US 2016/0122786 A1
(43) Pub. Date: May 5, 2016

(54) COMPOSITIONS AND METHODS FOR GENERATION OF BIOFUELS

(71) Applicant: THE REGENTS OF THE UNIVERSITY OF MICHIGAN, Ann Arbor, MI (US)

(72) Inventors: XIAOXIA LIN, ANN ARBOR, MI (US); MICHAEL NELSON, ANN ARBOR, MI (US); HENRY WANG, ANN ARBOR, MI (US); JEREMY MINTY, ANN ARBOR, MI (US); YANGXUE GAO, MILPITAS, CA (US); SARAH KISTLER, TROY, MI (US); DAVID BOYER, MUSKEGON, MI (US)

(21) Appl. No.: 14/931,107

(22) Filed: Nov. 3, 2015

Related U.S. Application Data
(60) Provisional application No. 62/075,609, filed on Nov. 5, 2014.

(51) Int. Cl. C12P 7/64; C10L 1/00; C12N 1/20

(52) U.S. Cl. CPC C12P 7/64; C12N 1/20; C10L 1/00; C10L 2200/0469; C10L 2290/26; C10L 2290/06

(57) ABSTRACT

Provided herein are compositions and method for generation of biofuels. In particular, provided herein are modified bacteria for use in processing intermediates of algae biomass processing.

This application claims the benefit of U.S. provisional application Ser. No. 62/075,609, filed Nov. 5, 2014, which is incorporated herein by reference in its entirety.

### STATEMENT REGARDING FEDERALLY SPONSORED RESEARCH OR DEVELOPMENT

This invention was made with government support under EFRI0937992 awarded by the National Science Foundation and 2012-67011-19723 awarded by the USDA/NIFA. The Government has certain rights in the invention.

### FIELD OF THE DISCLOSURE

Provided herein are compositions and methods for generation of biofuels. In particular, provided herein are modified bacteria for use in processing intermediates of algae biomass processing.

### BACKGROUND OF THE DISCLOSURE

All fossil fuels found in nature—petroleum, natural gas, and coal, based on biogenic hypothesis—are formed through processes of thermochemical conversion (TCC) from biomass buried beneath the ground and subjected to millions of years of high temperature and pressure. In particular, existing theories attribute that petroleum is from diatoms (algae) and deceased creatures and coal is from deposited plants. TCC is a chemical reforming process of biomass in a heated and usually pressurized, oxygen deprived enclosure, where long-chain organic compounds (solid biomass) break into short-chain hydrocarbons such as syngas or oil. TCC is a broad term that includes gasification, including the Fisher-Tropsch process, direct liquefaction, hydrothermal liquefaction, and pyrolysis. Gasification of biomass produces a mixture of hydrogen and carbon monoxide, commonly called syngas. The syngas is then reformed into liquid oil with the presence of a catalyst. Pyrolysis is a heating process of dried biomass to directly produce syngas and oil. Both gasification and pyrolysis require dried biomass as feedstock, and the processes occur in an environment higher than 600° C.

The hydrothermal liquefaction (HTL) involves direct liquefaction of biomass, with the presence of water and perhaps some catalysts, to directly convert biomass into liquid oil, with a reacting temperature of lower than 400° C.

Algae biomass is a promising feedstock for liquid biofuels. Hydrothermal liquefaction can convert wet algae biomass into a “biocrude” oil with similar properties to crude petroleum, but it also produces an aqueous co-product (AqA1) that contains most of the nitrogen and phosphorus from the initial biomass and up to 40% of the carbon. Efficient recycle of these components within the system is crucial, yet direct feeding of AqA1 back to algae ponds has proven problematic in many cases (Jena et al. 2011. Bioresource Technology 102:3, 3380-3387).

Methods of improving the efficiency and utilizing by-products of hydrothermal liquefaction of biomass are needed.

### SUMMARY OF THE DISCLOSURE

Provided herein are compositions and methods for generation of biofuels. In particular, provided herein are modified bacteria for use in processing intermediates of algae biomass processing.

For example, in some embodiments, the present disclosure provides an E. coli bacterium comprising one or more genomic mutations (e.g., including but not limited to t65318c, c85999t, g87397a, t3851043c, c1352926t, c1356347a, c1903524t, t2272940a, c2720457t, c2721067t, c2805823t, c2966838a, c3623658a, c4116935a, c4117061a, a4639705g, 2490773del.ttaa, 1972598ins.ggta, 1972598ins.ggt, 1974707ins.tcc, 1104288del.c, 1978024ins.attaccc, 682809ins.aagaaa, 2704495ins.t, 3804525ins.ca, or 3804339ins.c). In some embodiments, the bacterium comprises 1, 2, 3, 4, 5, or more of the mutations. In some embodiments, bacteria are engineered to have the one or more mutations. In some embodiments, the one or more genomic mutations encode an amino acid change selected from, for example, polB-ILE155VAL, ilvI-PRO124SER, ilvH-GLY14ASP14, ilvN-ASN17SER, sapD-GLY235SER, sapA-GLY255VAL, many-ALA148VAL, yejA-TYR193ASN, pka-GLN169 Stop, pka-SER372LEU, pro V-GLN337Stop, ptsP-GLU533Stop, yhhH-THR87ASN, glpK-LYS96ASN, glpK-TRP54CYS, or arcA-VAL201ALA. In some embodiments, the genomic mutation results in a stop codon, an insertion of an amino acid, or a frameshift in the amino acid sequence of the corresponding gene product. In some embodiments, the genomic mutation results in a non-functional gene product (e.g., a mutated protein, a truncated protein, or lack of synthesis of any gene product) from a gene selected from, for example, DNA polymerase II, Acetolactate Synthase Subunit I, Acetolactate Synthase Subunit H, Acetolactate Synthase Subunit N, Antimicrobial Peptide Transport D, Antimicrobial Peptide TransportA, Mannose-Specific PTS Component, Microsin C Transporter, Protein Lysine Acetyltransferase, Glycine Betaine Transporter Subunit, Fused PTS Enzyme, Hypothetical Protein yhhH, Glycerol Kinase, DNA-Binding Response Regulator, oxalyl CoA decarboxylase, ThDP-dependent, methyl-accepting chemotaxis protein II, fused chemotactic sensory histidine kinase in two-component regulatory system with CheB and CheY: sensory histidine kinase/signal sensing protein, curlin nucleator protein, minor subunit in curli complex, DNA-binding transcriptional dual regulator with F1hC, Hsp70 family chaperone Hsc62, binds to RpoD and inhibits transcription leader peptidase (signal peptidase I), or lipopolysaccharide core biosynthesis protein.

Additional embodiments provide a P. putida bacterium comprising one or more (e.g., 1, 2, 3, 4, 5, or more) genomic mutations selected from, for example, c3182744g, a3951158t, c1886927t, a1887316g, t1888040c, a4741121t, g4741135t, c4741365t, g1692767a, g1692775a, t1883248c, t1909387c, g5610571t, g318616a, g3114367a, g5610082t, c2901458g, 1499477ins.c, 1696422del.c, 1699726del.acct, 3183044del.g or 4842482del.cctgg. In some embodiments, the one or more genomic mutations encode an amino acid change selected from, for example, PP2793-PRO136ARG, PP3486-ILE22LYS, PP1695-GLY1115SER, PP1695-ILE985THR, PP1695-SER744GLY, PP1488-ALA290THR, PP0264-SER375LEU, PP2729-ARG224HIS, PP4929-ALA143GLU, or PP2554-ASP181GLU. In some embodiments, the genomic mutation results in a non-functional gene product from a gene selected from, for example, acyl-CoA dehydrogenase, cytochrome c, integral membrane sensor hybrid histidine kinase, (gltA) type II citrate synthase, methyl-accepting chemotaxis sensory transducer, hypothetical proteins PP_1690-PP_1691, fumarylacetoacetate hydrolase: major facilitator superfamily transporter phthalate permease, LysR family transcriptional regulator: small multidrug resistance protein, sensor histidine kinase, hypothetical protein PP_2729, LysR family transcriptional regulator, 4-hydroxyphenylpyruvate dioxygenase, tryptophanyl-tRNA synthetase: AFG1 family ATPase, chemotaxis protein CheA, response regulator/GGDEF domain-containing protein, or cytochrome c oxidase, cbb3-type subunit III.

Certain embodiments provide a population comprising substantially all (e.g., at least 80%, at least 90%, at least 95%, at least 99%, etc.) of one or more of the aforementioned bacteria. In some embodiments, a population of bacteria comprising at least 1 liter, at least 2 liters, at least 10 liters, at least 100 liters, etc. (e.g., in a reaction vessel or reactor) is provided. In some embodiments, a population comprising at least 200 million, 500 million, 1 billion or more bacteria is provided.

In some embodiments, the bacterium (e.g., the aforementioned bacterium) comprises a synthetic plasmid (e.g., comprising a marker or selectable marker).

Additional embodiments provide a composition comprising any of the aforementioned bacterium and a biomass (e.g., algae biomass). In some embodiments, the biomass is an aqueous co-product of algae biomass processes through hydrothermal liquefaction.

Yet other embodiments provide a system, comprising: a reaction vessel comprising any of the aforementioned bacterium and optionally a second reaction vessel comprising a biomass. In some embodiments, the system further comprises a temperature and pressure control component (e.g., configured to maintain a temperature of at least 200 deg C. and a pressure of at least 10 MPa in the reaction vessel for HTL or biomass or approximately 30° C. and atmospheric pressure for bacterial growth).

Still further embodiments provide a method of generating a biofuel, comprising: contacting aqueous co-product generated from a hydrothermal liquefaction reaction of biomass with any of the aforementioned bacterium under conditions such that the bacterium converts the aqueous co-product into a secondary biomass. In some embodiments, the method further comprises the step of subjecting the secondary biomass to hydrothermal liquefaction to generate a biofuel.

Additional embodiments are described herein.

### DESCRIPTION OF THE FIGURES

FIG. 1 shows an exemplary hydrothermal liquefaction process for bio-crude oil generation, with a microbial growth operation for supplemental biomass production.

FIG. 2 shows evolved strain growth over time in 30 vol % AqA1 medium.

FIG. 3 shows growth in AqA1 media of E. coli strains containing single SNP mutations compared to their parent strain.

### DEFINITIONS

To facilitate an understanding of the present disclosure, a number of terms and phrases are defined below:

As used herein, the term “biofuel” refers to any fuel that is derived from a biomass, e.g., recently living organisms, e.g., plants, or their metabolic byproducts, such as manure from cows. It is a renewable energy source, unlike other natural resources such as petroleum, coal and nuclear fuels.

The term “microorganism” includes prokaryotic and eukaryotic microbial species from the domains Archaea, Bacteria, and Eukarya, the latter including yeast and filamentous fungi, protozoa, algae, or higher Protista. The terms “microbial cells” and “microbes” are used interchangeably with the term “microorganism”.

The term “prokaryotes” refers to cells that contain no nucleus or other cell organelles. The prokaryotes are generally classified in one of two domains, the Bacteria and the Archaea. The definitive difference between organisms of the Archaea and Bacteria domains is based on fundamental differences in the nucleotide base sequence in the 16S ribosomal RNA.

The terms “bacteria” and “bacterium” and “archaea” and “archaeon” refer to prokaryotic organisms of the domain Bacteria and Archaea in the three-domain system (see Woese C R, et al., Proc Natl Acad Sci U S A 1990, 87: 4576-79).

The term “Bacteria” or “eubacteria” refers to a domain of prokaryotic organisms. Bacteria include at least 11 distinct groups as follows: (1) Gram-positive (gram+) bacteria, of which there are two major subdivisions: (1) high G+C group (Actinomycetes, Mycobacteria, Micrococcus, others) (2) low G+C group (Bacillus, Clostridia, Lactobacillus, Staphylococci, Streptococci, Mycoplasmas); (2) Proteobacteria, e.g., Purple photosynthetic+non-photosynthetic Gram-negative bacteria (includes most “common” Gram-negative bacteria); (3) Cyanobacteria, e.g., oxygenic phototrophs; (4) Spirochetes and related species; (5) Planctomyces; (6) Bacteroides, Flavobacteria; (7) Chlamydia; (8) Green sulfur bacteria; (9) Green non-sulfur bacteria (also anaerobic phototrophs); (10) Radioresistant micrococci and relatives; (11) Thermotoga and Thermosipho thermophiles.

The term “genus” is defined as a taxonomic group of related species according to the Taxonomic Outline of Bacteria and Archaea (Garrity et al. (2007) The Taxonomic Outline of Bacteria and Archaea. TOBA Release 7.7, March 2007. Michigan State University Board of Trustees).

The term “species” is defined as a collection of closely related organisms with greater than 97% 16S ribosomal RNA sequence homology and greater than 70% genomic hybridization and sufficiently different from all other organisms so as to be recognized as a distinct unit.

“Strain” as used herein in reference to a microorganism describes an isolate of a microorganism considered to be of the same species but with a unique genome and, if nucleotide changes are non-synonymous, a unique proteome differing from other strains of the same organism.

Strains may differ in their non-chromosomal genetic complement. Typically, strains are the result of isolation from a different host or at a different location and time, but multiple strains of the same organism may be isolated from the same host.

As used herein, the term “host cell”, “host microbial organism”, and “host microorganism” are used interchangeably to refer to any archaeal, bacterial, or eukaryotic living cell into which a heterologous entity (e.g., a biomolecule such as a nucleic acid, protein, etc.) can be, or has been, inserted. The term also relates to the progeny of the original cell, which may not be completely identical in morphology or in genomic or total DNA complement to the original parent, due to natural, accidental, or deliberate mutation.

The terms “modified microorganism,” “recombinant microorganism”, and “recombinant host cell” refer to a non-naturally occurring organism that is produced by methods such as inserting, expressing, or overexpressing endogenous polynucleotides; by expressing or overexpressing heterologous polynucleotides, such as those included in a vector; by introducing a mutation into the microorganism; or by altering the expression of an endogenous gene. In embodiments relating to the introduction of a polynucleotide into a microorganism, the polynucleotide generally encodes a target enzyme involved in a metabolic pathway for producing a desired metabolite. It is understood that the terms “recombinant microorganism” and “recombinant host cell” refer not only to the particular recombinant microorganism but to the progeny or potential progeny of such a microorganism. Because certain modifications may occur in succeeding generations due to either mutation or environmental influences, such progeny may not, in fact, be identical to the parent cell, but are still included within the scope of the term as used herein.

The term “wild-type microorganism” describes a cell that occurs in nature, e.g., a cell that has not been genetically modified. A wild-type microorganism can be genetically modified to express or overexpress a target enzyme. This microorganism can act as a parental microorganism in the generation of a microorganism modified to express or overexpress a target enzyme. In turn, the microorganism modified to express or overexpress one or more target enzymes can be modified to express or overexpress another target enzyme.

Accordingly, a “parental microorganism” functions as a reference cell for successive genetic modification events. Each modification event can be accomplished by introducing a nucleic acid molecule into the reference cell. The introduction facilitates the expression or overexpression of a target enzyme. It is understood that the term “facilitates” encompasses the activation of endogenous polynucleotides encoding a target enzyme through genetic modification of, e.g., a promoter sequence in a parental microorganism. It is further understood that the term “facilitates” encompasses the introduction of heterologous polynucleotides encoding a target enzyme in to a parental microorganism.

The term “mutation” as used herein indicates any modification of a nucleic acid that results in an altered nucleic acid, e.g., that produces an amino acid “substitution” in a polypeptide (e.g., thus producing a “mutant” polypeptide or “mutant” nucleic acid). Mutations include, for example, point mutations, deletions, or insertions of single or multiple residues in a polynucleotide, which includes alterations arising within a protein-encoding region of a gene as well as alterations in regions outside of a protein-encoding sequence, such as, but not limited to, regulatory or promoter sequences. A genetic alteration may be a mutation of any type. For instance, the mutation may constitute a point mutation, a frame-shift mutation, an insertion, or a deletion of part or all of a gene. In addition, in some embodiments of the modified microorganism, a portion of the microorganism genome has been replaced with a heterologous polynucleotide. In some embodiments, the mutations are naturally-occurring. In other embodiments, the mutations are the results of artificial mutation pressure. In still other embodiments, the mutations in the microorganism genome are the result of genetic engineering.

As used herein, the term “hydrothermal liquefaction” or “pyrolysis” refers to the thermochemical decomposition of organic material at elevated temperatures in the absence of oxygen. In general, pyrolysis of organic substances produces gas and liquid products (e.g., biofuel) and leaves a solid residue richer in carbon content (e.g., biochar). In some embodiments, biomass (e.g., algae biomass) serves as a starting material for hydrothermal liquefaction (See e.g., U.S. Pat. No. 8,759,068).

As used herein, the term “aqueous co-product” refers to an aqueous product of the hydrothermal liquefaction of biomass.

As used herein, the term “nucleic acid molecule” refers to any nucleic acid containing molecule, including but not limited to, DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide, precursor, or RNA (e.g., rRNA, tRNA). The polypeptide can be encoded by a full length coding sequence or by any portion of the coding sequence so long as the desired activity or functional properties (e.g., enzymatic activity, ligand binding, signal transduction, immunogenicity, etc.) of the full-length or fragments are retained. The term also encompasses the coding region of a structural gene and the sequences located adjacent to the coding region on both the 5′ and 3′ ends for a distance of about 1 kb or more on either end such that the gene corresponds to the length of the full-length mRNA. Sequences located 5′ of the coding region and present on the mRNA are referred to as 5′ non-translated sequences. Sequences located 3′ or downstream of the coding region and present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

The term “protein” or “polypeptide” as used herein indicates an organic polymer composed of two or more amino acidic monomers and/or analogs thereof. As used herein, the term “amino acid” or “amino acidic monomer” refers to any natural and/or synthetic amino acids including glycine and both D or L optical isomers. The term “amino acid analog” refers to an amino acid in which one or more individual atoms have been replaced, either with a different atom, or with a different functional group. Accordingly, the term polypeptide includes amino acidic polymer of any length including full length proteins, and peptides as well as analogs and fragments thereof. A polypeptide of three or more amino acids is also called a protein oligomer or oligopeptide

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

As used herein, the term “sample” is used in its broadest sense. In one sense, it is meant to include a specimen or culture obtained from any source, as well as biological and environmental samples. Biological samples may be obtained from animals (including humans) and encompass fluids, solids, tissues, and gases. Biological samples include blood products, such as plasma, serum and the like. Environmental samples include, but are not limited to, biomass, products of hydrothermal liquefaction of biomass, etc. Such examples are not however to be construed as limiting the sample types applicable to the present disclosure.

### DETAILED DESCRIPTION OF THE DISCLOSURE

Provided herein are compositions and methods for generation of biofuels. In particular, provided herein are modified bacteria for use in processing intermediates of algae biomass processing.

Hydrothermal liquefaction (HTL), also called hydrous pyrolysis, is a process for the reduction of complex organic materials such as bio-waste or biomass into crude oil and other chemicals. HTL has different pathways for the biomass feedstock. Unlike biological treatment such as anaerobic digestion, HTL converts feedstock into oil rather than gases or alcohol. There are some unique features of the HTL process and its product compared with other biological processes. First, the end product is crude oil which has a much higher energy content than syngas or alcohol. And second, if the feedstock contains a lot of water, HTL does not require drying as gasification or pyrolysis. The drying process typically takes large quantities of energy and time. The energy used to heat up the feedstock in the HTL process could be recovered effectively with the existing technology.

HTL may have two pathways from biomass to biofuel: (1) direct conversion of biomass or (2) pretreatment of biomass and then fermentation. For biomass with little lignocellulosic fraction—such as waste streams from animal, human, and food processing—it can be directly converted into biofuel thermochemically.

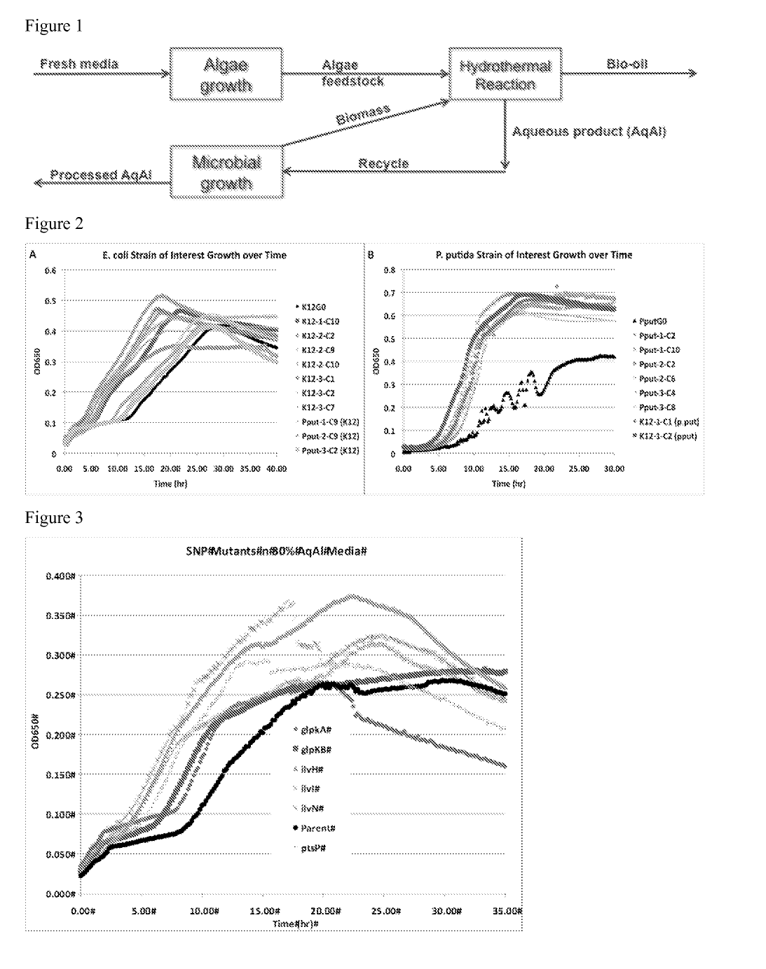

HTL of biomass such as algae biomass generates an aqueous co-product intermediate (See e.g., FIG. 1). An intermediate bacterial culture may be able to utilize this challenging material stream as an energy and nutrient source. This culture could improve the overall efficiency of the integrated biorefinery by generating a supplemental biomass source for the hydrothermal reaction (FIG. 1).

Initial studies into bacterial growth on AqA1 have shown that Escherichia coli and Pseudomonas putida bacteria can withstand higher concentrations of AqA1 than algae cultures can. However, there is still significant toxic effect and the bacteria can only metabolize, at best, half of the organic carbon present in the AqA1 (Nelson et al. 2013. Bioresource echnology. 136, 522-528).

Provided herein are modified E. coli and P. putida that confer better growth characteristics in AqA1 media when compared to natural strains. The engineered bacteria described herein find use in a variety of applications.

The present disclosure is not limited to particular mutations. Mutations are one or more of single nucleotide changes, insertions of one or more nucleotides, or deletions of one or more nucleotides. Mutations or combinations of mutations are made in one or more genes of the bacterium. Exemplary mutations of Escherichia coli and Pseudomonas putida are described in Tables 1-4. In some embodiments, Escherichia coli and Pseudomonas putida are engineered to have 1, 2, 3, 4, or more of the mutations described in Tables 1-4.

The accession numbers for both genomes are as follows:

DEFINITION Escherichia coli str. K-12 substr. MG1655, complete genome.

### ACCESSION U00096

### VERSION U00096.3 GI:545778205 and

DEFINITION Pseudomonas putida KT2440 complete genome.

### ACCESSION AE015451 AE016774-AE016794

### VERSION AE015451.1 GI:24987239

Numbers indicating the location of a mutation (e.g. “t65318c”) are in reference to the nucleotide number in the E. coli MG1655 genome sequence (Accession #U00096, Version U00096.3) or P. putida KT2440 genome sequence (Accession AE015451, Version AE015451.1), available publicly from NCBI. In variants of these two organisms, or in genes derived from these organisms and inserted into others, the mutations are those in the gene or space between two genes that correspond to the locations in the whole genome sequence versions stated previously.

For amino acid sequences, numbering is based on the amino acid sequence for the given gene (See above accession numbers).

As used herein, the term “t65381c” and the like refers to a change from a t nucleotide to a c nucleotide at position 65381 of the corresponding nucleic acid sequence. The term “65381ins.tt” refers to the insertion of two t nucleotides at position 65381 of the corresponding sequence. Likewise, 65381del.tt refers to a deletion of two t nucleotides at position 65381 of the corresponding sequence. The term “ilvH-GLY14ASP14” refers to the substitution of an ASP for a GLY at position 14 of the ilvH gene.

Examples of specific mutations include, but are not limited to, E. coli mutations t65318c, c85999t, g87397a, t3851043c, c1352926t, c1356347a, c1903524t, t2272940a, c2720457t, c2721067t, c2805823t, c2966838a, c3623658a, c4116935a, c4117061a, a4639705g, 2490773del.ttaa, 1972598ins.ggta, 1972598ins.ggt, 1974707ins.tcc, 1104288del.c, 1978024ins.attaccc, 682809ins.aagaaa, 2704495ins.t, 3804525ins.ca, or 3804339ins.c and P. putida mutations c3182744g, a3951158t, c1886927t, a1887316g, t1888040c, a4741121t, g4741135t, c4741365t, g1692767a, g1692775a, t1883248c, t1909387c, g5610571t, g318616a, g3114367a, g5610082t, c2901458g, 1499477ins.c, 1696422del.c, 1699726del.acct, 3183044del.g or 4842482del.cctgg.

In some embodiments, mutations result in altered (e.g., mutated or truncated) polypeptides. In some embodiments, mutations result in the introduction of a frameshift or stop codon that truncates a protein encoded by the gene. In some embodiments, the polypeptide expressed by a mutated gene is non-functional, has increased function, or is not expressed.

Exemplary amino acid mutations include, for example, 1 or more of polB-ILE155VAL, ilvI-PRO124SER, ilvH-GLY14ASP14, ilvN-ASN17SER, sapD-GLY235SER, sapA-GLY255VAL, many-ALA148VAL, yejA-TYR193ASN, pka-GLN169 Stop, pka-SER372LEU, pro V-GLN337Stop, ptsP-GLU533Stop, yhhH-THR87ASN, glpK-LYS96ASN, glpK-TRP54CYS, or arcA-VAL201ALA mutations in E. coli and PP2793-PRO136ARG, PP3486-ILE22LYS, PP1695-GLY1115SER, PP1695-ILE985THR, PP1695-SER744GLY, PP1488-ALA290THR, PP0264-SER375LEU, PP2729-ARG224HIS, PP4929-ALA143GLU, or PP2554-ASP181GLU mutations in P. putida.

Variant P. putida and/or E. coli bacteria are generated using any suitable method. Variants may have improved function and biological activity (e.g., improved growth in AqA1) than the parent (or wild-type) protein. Due to redundancy in the genetic code, nucleic acid variants may or may not affect amino acid sequence. A nucleic acid variant may also encode an amino acid sequence comprising one or more conservative substitutions compared to a reference amino acid sequence. A conservative substitution may occur naturally in the polypeptide (e.g., naturally occurring genetic variants) or may be introduced when the polypeptide is recombinantly produced. A conservative substitution is where one amino acid is substituted for another amino acid that has similar properties, such that one skilled in the art would expect that the secondary structure and hydropathic nature of the polypeptide to be substantially unchanged. Amino acid substitutions may generally be made on the basis of similarity in polarity, charge, solubility, hydrophobicity, and/or the amphipathic nature of the residues, and is known in the art. Amino acid substitutions, deletions, and additions may be introduced into a polypeptide using well-known and routinely practiced mutagenesis methods (see, e.g., Sambrook et al. Molecular Cloning: A Laboratory Manual, 3d ed., Cold Spring Harbor Laboratory Press, NY 2001).

Oligonucleotide-directed site-specific (or segment specific) mutagenesis procedures may be employed to provide an altered polynucleotide that has particular codons altered according to the substitution, deletion, or insertion desired. Deletion or truncation variants of proteins may also be constructed by using convenient restriction endonuclease sites adjacent to the desired deletion. Alternatively, random mutagenesis techniques, such as alanine scanning mutagenesis, error prone polymerase chain reaction mutagenesis, and oligonucleotide-directed mutagenesis may be used to prepare polypeptide variants (see, e.g., Sambrook et al, supra). Nucleic acids encoding an enzyme with coenzyme M reductase activity may be combined with other nucleic acid sequences, such as promoters, polyadenylation signals, restriction enzyme sites, multiple cloning sites, other coding segments, and the like.

Differences between a wild-type (or parent) nucleic acid or polypeptide and the variant thereof, may be determined by suitable methods (e.g., those described in Example 1). Methods to determine sequence identity can be applied from publicly available computer programs. Computer program methods to determine identity between two sequences include, for example, BLASTP, BLASTN (Altschul, S. F. et al, J. Mol. Biol. 215: 403-410 (1990), and FASTA (Pearson and Lipman Proc. Natl. Acad. Sci. USA 85; 2444-2448 (1988). The BLAST family of programs is publicly available from NCBI and other sources (BLAST Manual, Altschul, S., et al, NCBI NLM NIH Bethesda, Md.

Assays for determining whether a polypeptide variant folds into a conformation comparable to the non-variant polypeptide or fragment include, for example, the ability of the protein to react with mono- or polyclonal antibodies that are specific for native or unfolded epitopes, the retention of ligand-binding functions, the retention of enzymatic activity (if applicable), and the sensitivity or resistance of the mutant protein to digestion with proteases (see Sambrook et al, supra). Polypeptides, variants and fragments thereof, can be prepared without altering a biological activity of the resulting protein molecule (e.g., without altering one or more functional activities in a statistically significant or biologically significant manner). For example, such substitutions are generally made by interchanging an amino acid with another amino acid that is included within the same group, such as the group of polar residues, charged residues, hydrophobic residues, and/or small residues, and the like. The effect of any amino acid substitution may be determined empirically merely by testing the resulting modified protein for the ability to function in a biological assay, or to bind to a cognate ligand or target molecule.

In some embodiments, variant strains of P. putida and E. coli comprising one or more artificial traits (e.g., added via a synthetic plasmid). For example, in some embodiments, bacteria comprise selectable or screenable markers to control for contamination by other bacteria and/or genetic drift. Exemplary selectable and/or screenable markers include, but are not limited to, antibiotic resistance genes, genes that permit or restrict growth in the presence of certain nutrients, beta-galactosidase genes, and green fluorescent protein.

In some embodiments, variant strains of P. putida and E. coli are generated by directed evolution methods (See e.g., Example 1).

Embodiments of the present disclosure provide compositions, kits and systems comprising variant (e.g., engineered) strains of P. putida and E. coli such as those described herein. In some embodiments, compositions, kits, and systems comprise substrates (e.g., biomass, algae biomass, and/or AqA1 or other aqueous co or by-products of hydrothermal liquefaction), reaction vessels, temperature control components, reagents for monitoring reactions, etc. Hydrothermal liquefaction is typically performed at high temperatures and pressures (e.g., a temperature of at least 200 deg C. and a pressure of at least 10 MPa in the reaction vessel). Treatment with bacteria (e.g., fermentation of AqA1) is typically carried out at approximately 30° C. and atmospheric pressure. Thus, in some embodiments, reaction vessels comprise temperature and pressure controls and sensors configured to maintain suitable temperature and pressure for reactions. In some embodiments, systems comprise multiple reactors (e.g., HTL reactors and bacterial growth reactors). In some embodiments, systems comprise transport components to move aqueous co-products form HTL reactors to bacterial reactors and then return additional biomass to a HTL reactor or other component of the system.

In some embodiments, commercially available bioreactor systems are utilized. Examplary systems include, but are not limited to, those available from GE Healthcare Life Sciences, Pittsburgh, Pa.; Membrane Bioreactor (MBR) Systems from Evoqua, Alpharetta, Ga.; and technology from Total American Services, Inc., Houston, Tex.

In some embodiments, bioreactor systems comprise computer software and a computure processor (e.g., to control temperature and pressure of reactors, move components from one reactor to another, remove biofuel generated in the reactor, etc.).

The variant strains of P. putida and E. coli described herein find use in a variety of applications. In some embodiments, variant strains of P. putida and E. coli are used in generation of biofuels from biomass (e.g., algae biomass, although other types of biomass can be utilized). In some embodiments, the strains are used to generate additional biomass from hydrothermal liquefaction by-products such as AqA1. For example, in some embodiments, a first reaction (e.g., HTL) utilizes biomass to generate biofuel and AqA1. Such AqA1 by-products are converted by the bacteria described herein into additional biomass that can then be recycled into the hydrothermal liquefaction reaction or another reaction to generate fuel (e.g., as shown in FIG. 1). In some embodiments, AqA1 is removed from the HTL reaction vessel or component, treated with bacteria, and sent to an additional biorefinery process component.

In some embodiments, variant strains of P. putida and E. coli described herein find use in additional research and industrial uses (e.g., research into improved methods of generating biofuel). For example, in some embodiments, variant bacteria are engineered or otherwise generated, their growth in the presence of AqA1 is assayed, and variant bacteria that grow in the presence of AqA1 are selected for further analysis. In some embodiments, variant bacteria that exhibit increased growth in the presence of AqA1 are screened for their ability to generate biomass and/or undergo sequencing analysis to confirm the presence of one or more nucleic acid variants.

### EXPERIMENTAL

The following examples are provided in order to demonstrate and further illustrate certain preferred embodiments and aspects of the present disclosure and are not to be construed as limiting the scope thereof.

### Example 1

An adaptive evolution study was carried out where E. coli MG1655 and P. putida KT2440 were grown in media containing AqA1 as the sole carbon, nitrogen, and phosphorus source. Small amounts of cell culture were transferred to fresh media every two to three days, keeping the cells in a state of constant growth and reproduction. The AqA1 concentration was increased when substantial growth improvement was observed. This adaptive evolution process was carried out for roughly 300 generations of cell growth and division. During this time, natural mutagenesis randomly created mutations in cells' DNA with a small frequency per cell division, and mutants that better tolerated AqA1 grew faster and became the dominant fraction of the culture (natural selection).

After the conclusion of this evolution procedure, individual, isogenic strains were isolated that could grow faster and to higher cell densities (yields) in AqA1 growth medium when compared to their un-evolved “parent” strains. FIG. 2 shows these strains' growth in AqA1 media in comparison to the parent they evolved from. Most strains outperform their parent by having a shorter lag time before growth, a faster growth rate, and/or an increased maximum cell density.

The entire genomes of each isolated strain were sequenced and compared to parent genomes. Single nucleotide polymorphisms (SNPs) and small nucleotide insertions/deletions (indels) responsible for improved growth performance were determined.

Mutations are shown in Tables 1-4. Tables 1 (for P. putida) and 2 (for E. coli) are single nucleotide polymorphisms (SNP) indicating individual nucleotide replacement mutations. The gene name and function, along with the exact location in the genome the mutation occurs, and what nucleotide the mutant substitutes for the initial parent (WT) nucleotide are shown. The immediate effects of these SNP mutations can be classified into three categories: i) for a majority of them, each SNP causes a change in a single amino acid residue in the protein the gene codes for; ii) a SNP causes the corresponding protein to terminate prematurely by changing an amino-acid-coding codon to a stop codon; and iii) a SNP occurs in an inter-genic region, which may affect the regulation and hence expression level of certain genes locally or globally.

Tables 3 (for P. putida) and 4 (for E. coli) are small insertion/deletion (indel) mutations. These involve either the inclusion of extra nucleotides or their removal at certain positions in the genome (removals are indicated with a “−” sign in the “indel” column of the tables). Similar to a SNP described above, an indel in the protein coding region can also introduce a premature stop codon. More often, an indel in the protein coding region changes the residue sequence. If the number of inserted or deleted nucleotides is a multiple of three, the resulted protein has a small region in the middle modified (addition/deletion/change of amino acid residues, depending on the exact location of the indel). Otherwise, an indel would cause a frameshift, changing the residue sequence starting from the insertion/deletion point and lead to inactivation of the protein function.

TABLE 1
Single Nucleotide Polymorphism mutations from P. putida mutants

| Gene Name | Gene Function | Location | WT | Mutant | Residue Change |
|---|---|---|---|---|---|
| PP_2793 | acyl-CoA dehydrogenase | 3182744 | C | G | PRO-136 => ARG-136 |
| PP_3486 | cytochrome c | 3951158 | A | T | ILE-22 => LYS-22 |
| PP_1695 | integral membrane | 1886927 | C | T | GLY-1115 => SER-1115 |
| | sensor hybrid histidine | 1887316 | A | G | ILE-985 => THR-985 |
| | kinase | 1888040 | T | C | SER-744 => GLY-744 |
| gltA-PP_4195 | (gltA) type II citrate | 4741121 | A | T | — |
| | synthase: hypothetical | 4741135 | G | T | — |
| | protein | 4741365 | C | T | — |
| PP_1488 | methyl-accepting | 1692767 | G | A | ALA-290 => THR-290 |
| | chemotaxis sensory | 1692775 | G | A | GLU-292 => GLU-292 |
| | transducer | | | | |
| PP_1690-PP_1691 | hypothetical proteins | 1883248 | T | C | — |
| PP_1709-PP_1710 | fumarylacetoacetate | 1909387 | T | C | — |
| | hydrolase: major | | | | |
| | facilitator superfamily | | | | |
| | transporter phthalate | | | | |
| | permease | | | | |
| PP_4929-PP_4930 | LysR family | 5610571 | G | T | — |
| | transcriptional regulator: | | | | |
| | small multidrug | | | | |
| | resistance protein | | | | |
| PP_0264 | sensor histidine kinase | 318616 | G | A | SER-375 => LEU-375 |
| PP_2729 | hypothetical protein | 3114367 | G | A | ARG-224 => HIS-224 |
| PP_4929 | LysR family | 5610082 | G | T | ALA-143 => GLU-143 |
| | transcriptional regulator | | | | |
| PP_2554 | 4- | 2901458 | C | G | ASP-181 => GLU-181 |
| | hydroxyphenylpyruvate | | | | |
| | dioxygenase | | | | |

TABLE 2
Single Nucleotide Polymorphism mutations from E. coli mutants

| Gene | | | | | |
|---|---|---|---|---|---|
| Name | Gene Function | Location | WT | Mutant | Residue Change |
| polB | DNA polymerase II | 65318 | T | C | ILE-155 => VAL-155 |
| ilvI | Acetolactate Synthase | 85999 | C | T | PRO-124 => SER-124 |
| | Subunit | | | | |
| ilvH | Acetolactate Synthase | 87397 | G | A | GLY-14 => ASP-14 |
| | Subunit | | | | |
| ilvN | Acetolactate Synthase | 3851043 | T | C | ASN-17 => SER-17 |
| | Subunit | | | | |
| sapD | Antimicrobial Peptide | 1352926 | C | T | GLY-235 => SER-235 |
| | Transport | | | | |
| sapA | Antimicrobial Peptide | 1356347 | C | A | GLY-255 => VAL-255 |
| | Transport | | | | |
| manY | Mannose-Specific PTS | 1903524 | C | T | ALA-148 => VAL-148 |
| | Component | | | | |
| yejA | Microsin C Transporter | 2272940 | T | A | TYR-193 => ASN-193 |
| pka | Protein Lysine | 2720457 | C | T | GLN-169 => Stop-169 |
| | Acetyltransferase | 2721067 | C | T | SER-372 => LEU-372 |
| proV | Glycine Betaine | 2805823 | C | T | GLN-337 => Stop-337 |
| | Transporter Subunit | | | | |
| ptsP | Fused PTS Enzyme | 2966838 | C | A | GLU-533 => Stop-533 |
| yhhH | Hypothetical Protein | 3623658 | C | A | THR-87 => ASN-87 |
| glpK | Glycerol Kinase | 4116935 | C | A | LYS-96 => ASN-96 |
| | | 4117061 | C | A | TRP-54 => CYS-54 |
| arcA | DNA-Binding Response | 4639705 | A | G | VAL-201 => ALA-201 |
| | Regulator | | | | |

TABLE 3
Small insertion/deletion mutations from P. putida mutants

| | position in | pos in | | | |
|---|---|---|---|---|---|
| gene | genome | gene | definition | indel | effect |
| PP_1311-PP_1312 | 1499477 | NA | tryptophanyl-tRNA synthetase: | 1:C | NA |
| | | | AFG1 family ATPase | | |
| PP_1492 | 1696422 | 458 | chemotaxis protein CheA | −1:C | frameshift |
| PP_1494 | 1699726 | 415 | response regulator/GGDEF domain- | −4:ACCT | stop |
| | | | containing protein | | |
| PP_2793 | 3183044 | 706 | acyl-CoA dehydrogenase | −1:G | frameshift |
| ccop-2 | 4842482 | NA | cytochrome c oxidase, cbb3-type | −5:CCTGG | NA |
| | | | subunit III | | |

TABLE 4
Small insertion/deletion mutations from E. coli mutants

| gene | position in | position in | | | |
|---|---|---|---|---|---|
| name | genome | gene | Gene Definition | indel | Effect |
| oxc | 2490773 | 1177 | oxalyl CoA decarboxylase, | −4:TTAA | frameshift |
| | | | ThDP-dependent | | |
| tar | 1972598 | 93 | methyl-accepting chemotaxis | 4:GGTA | frameshift |
| | | | protein II | | |
| tar | 1972598 | 93 | methyl-accepting chemotaxis | 3:GGT | extra |
| | | | protein II | | residue |
| cheA | 1974707 | 617 | fused chemotactic sensory | 3:TCC | altered |
| | | | histidine kinase in two- | | residue + |
| | | | component regulatory system | | extra |
| | | | with CheB and CheY: sensory | | residue |
| | | | histidine kinase/signal sensing | | |
| | | | protein | | |
| csgB | 1104288 | 337 | curlin nucleator protein, minor | −1:C | frameshift |
| | | | subunit in curli complex | | |
| flhD | 1978024 | 173 | DNA-binding transcriptional | 7:ATTACCC | frameshift |
| | | | dual regulator with FlhC | | |
| hscC | 682809 | 584 | Hsp70 family chaperone Hsc62, | 6:AAGAAA | altered |
| | | | binds to RpoD and inhibits | | residue + |
| | | | transcription | | extra |
| | | | | | residue |
| lepB | 2704495 | 814 | leader peptidase (signal | 1:T | frameshift |
| | | | peptidase I) | | |
| waaS | 3804525 | 591 | lipopolysaccharide core | 2:CA | frameshift |
| | | | biosynthesis protein | | |
| waaS | 3804339 | 777 | lipopolysaccharide core | 1:C | frameshift |
| | | | biosynthesis protein | | |

All publications, patents, patent applications and accession numbers mentioned in the above specification are herein incorporated by reference in their entirety. Although the disclosure has been described in connection with specific embodiments, it should be understood that the disclosure as claimed should not be unduly limited to such specific embodiments. Indeed, various modifications and variations of the described compositions and methods of the disclosure will be apparent to those of ordinary skill in the art and are intended to be within the scope of the following claims.

We claim:

1. An E. coli bacterium comprising one or more genomic mutations selected from the group consisting of t65318c, c85999t, g87397a, t3851043c, c1352926t, c1356347a, c1903524t, t2272940a, c2720457t, c2721067t, c2805823t, c2966838a, c3623658a, c4116935a, c4117061a, a4639705g, 2490773del.ttaa, 1972598ins.ggta, 1972598ins.ggt, 1974707ins.tcc, 1104288del.c, 1978024ins.attaccc, 682809ins.aagaaa, 2704495ins.t, 3804525ins.ca, and 3804339ins.c.

2. The E. coli bacterium of claim 1, wherein said one or more genomic mutations are two or more genomic mutations.

3. The E. coli bacterium of claim 1, wherein said one or more genomic mutations are three or more genomic mutations.

4. The E. coli bacterium of claim 1, wherein said one or more genomic mutations are five or more genomic mutations.

5. The E. coli bacterium of claim 1, wherein said one or more genomic mutations encode an amino acid change selected from the group consisting of polB-ILE155VAL, ilvI-PRO124SER, ilvH-GLY14ASP14, ilvN-ASN17SER, sapD-GLY235SER, sapA-GLY255VAL, many-ALA148VAL, yejA-TYR193ASN, pka-GLN169 Stop, pka-SER372LEU, pro V-GLN337Stop, ptsP-GLU533Stop, yhhH-THR87ASN, glpK-LYS96ASN, glpK-TRP54CYS, or arcA-VAL201ALA.

6. The E. coli bacterium of claim 1, wherein said genomic mutation results in a stop codon, an insertion of an amino acid, or a frameshift in amino acid sequences encoded by said genomic mutation.

7. The E. coli bacterium of claim 1, wherein said genomic mutation results in a non-functional gene product from a gene selected from the group consisting of DNA polymerase II, Acetolactate Synthase Subunit I, Acetolactate Synthase Subunit H, Acetolactate Synthase Subunit N, Antimicrobial Peptide Transport D, Antimicrobial Peptide TransportA, Mannose-Specific PTS Component, Microsin C Transporter, Protein Lysine Acetyltransferase, Glycine Betaine Transporter Subunit, Fused PTS Enzyme, Hypothetical Protein yhhH, Glycerol Kinase, DNA-Binding Response Regulator, oxalyl CoA decarboxylase, ThDP-dependent, methyl-accepting chemotaxis protein II, fused chemotactic sensory histidine kinase in two-component regulatory system with CheB and CheY: sensory histidine kinase/signal sensing protein, curlin nucleator protein, minor subunit in curli complex, DNA-binding transcriptional dual regulator with F1hC, Hsp70 family chaperone Hsc62, binds to RpoD and inhibits transcription leader peptidase (signal peptidase I), and lipopolysaccharide core biosynthesis protein.

8. The E. coli bacterium of claim 7, wherein said non-functional gene product is selected from the group consisting of a mutated protein, a truncated protein, or lack of synthesis of any gene product.

9. The E. coli bacterium of claim 1, wherein said bacterium further comprises an artificial plasmid.

10. The E. coli bacterium of claim 9, wherein said artificial plasmid comprises a selectable or screenable marker.

11. A P. putida bacterium comprising one or more genomic mutations selected from the group consisting of c3182744g, a3951158t, c1886927t, a1887316g, t1888040c, a4741121t, g4741135t, c4741365t, g1692767a, g1692775a, t1883248c, t1909387c, g5610571t, g318616a, g3114367a, g5610082t, c2901458g, 1499477ins.c, 1696422del.c, 1699726del.acct, 3183044del.g and 4842482del.cctgg.

12. The P. putida bacterium of claim 11, wherein said one or more genomic mutations are two or more genomic mutations.

13. The P. putida bacterium of claim 11, wherein said one or more genomic mutations are three or more genomic mutations.

14. The P. putida bacterium of claim 11, wherein said one or more genomic mutations are five or more genomic mutations.

15. The P. putida bacterium of claim 11, wherein said one or more genomic mutations encode an amino acid change selected from the group consisting of PP2793-PRO136ARG, PP3486-ILE22LYS, PP1695-GLY1115SER, PP1695-ILE985THR, PP1695-SER744GLY, PP1488-ALA290THR, PP0264-SER375LEU, PP2729-ARG224HIS, PP4929-ALA143GLU, or PP2554-ASP181GLU.

16. The P. putida bacterium of claim 11, wherein said genomic mutation results in a stop codon or frameshift in an amino acid sequences encoded by said genomic mutation.

17. The P. putida bacterium of claim 11, wherein said genomic mutation results in a non-functional gene product from a gene selected from the group consisting of acyl-CoA dehydrogenase, cytochrome c, integral membrane sensor hybrid histidine kinase, (gltA) type II citrate synthase, methyl-accepting chemotaxis sensory transducer, hypothetical proteins PP_1690-PP_1691, fumarylacetoacetate hydrolase: major facilitator superfamily transporter phthalate permease, LysR family transcriptional regulator: small multidrug resistance protein, sensor histidine kinase, hypothetical protein PP_2729, LysR family transcriptional regulator, 4-hydroxyphenylpyruvate dioxygenase, tryptophanyl-tRNA synthetase: AFG1 family ATPase, chemotaxis protein CheA, response regulator/GGDEF domain-containing protein, and cytochrome c oxidase, cbb3-type subunit III.

18. A method of generating a biofuel, comprising:
contacting aqueous co-product generated from a hydrothermal liquefaction reaction of biomass with the bacterium of claim 1 under conditions such that said bacterium converts said aqueous co-product into a secondary biomass.

19. The method of claim 18, further comprising the step of subjecting said secondary biomass to hydrothermal liquefaction to generate a biofuel.

20. The method of claim 19, wherein said biomass is an algae biomass.
